(19) United States
(12) Patent Application Publication
(10) Pub. No.: US 2013/0226187 A1
(43) Pub. Date: Aug. 29, 2013

(54) DEVICES AND METHODS FOR INJECTING FLUID INTO THE BODY

(71) Applicants: Laurent Schaller, Los Altos, CA (US); David S. Needleman, San Carlos, CA (US); Timothy J. McGrath, Fremont, CA (US); Ryan Connolly, Redwood City, CA (US)

(72) Inventors: Laurent Schaller, Los Altos, CA (US); David S. Needleman, San Carlos, CA (US); Timothy J. McGrath, Fremont, CA (US); Ryan Connolly, Redwood City, CA (US)

(73) Assignee: Benvenue Medical, Inc., Santa Clara (US)

(21) Appl. No.: 13/878,081

(22) PCT Filed: Oct. 7, 2011
(86) PCT No.: PCT/US11/55207

§ 371 Date: May 6, 2013

Related U.S. Application Data
(60) Provisional application No. 61/390,812, filed on Oct. 7, 2010.
(60) Provisional application No. 61/411,554, filed on Nov. 9, 2010.
(60) Provisional application No. 61/500,929, filed on Jun. 24, 2011.

(51) Int. Cl. A61B 17/88

(52) U.S. Cl. CPC A61B 17/8816
USPC 606/92

(57) ABSTRACT

Devices are provided for injecting fluid into the body. One such device includes an elongated, hollow cannula having a proximal end portion, a distal end portion, a shaft portion between the proximal and distal end portions, and a fluid flow lumen extending through the shaft portion between the proximal and distal end portions. The distal end portion is flexible and pre-disposed to assume a curved configuration having a radially inner side and a radially outer side. The radially inner side includes at least one aperture to direct fluid flowing from the lumen in a radially inward direction. The device may include an introducer member and/or a guide member for introducing the cannula to a treatment site. Methods of injecting fluid into the body are also provided.

### CROSS REFERENCE TO RELATED APPLICATIONS

This application claims the benefit of U.S. Provisional Application Ser. No. 61/390,812, filed Oct. 7, 2010, U.S. Provisional Application Ser. No. 61/411,554, filed Nov. 9, 2010 and U.S. Provisional Application Ser. No. 61/500,929, filed Jun. 24, 2011, all of which are hereby incorporated by reference in their entireties.

### TECHNICAL FIELD

The present disclosure generally relates to devices and methods employed in minimally invasive surgical procedures. More particularly, the present disclosure generally relates to various devices and methods for injecting fluid into a treatment site within a patient, especially bones including vertebrae for the treatment of compression fractures.

### BACKGROUND ART

It is often necessary or desirable to administer, deliver or inject fluid to a particular target location or zone in the human body for therapeutic or diagnostic treatments or procedures. This can include a variety of different fluids, including orthopedic cements and other bone augmentation materials, drugs, contrast agents, cell-based treatment materials such as stem cells, and other fluids, and for a variety of different diagnostic or therapeutic treatments or procedures. Although the subject matter of this disclosure is not limited to orthopedic treatments in general or spinal treatments in particular. Treatment of spinal conditions, including treatment of compression fractures of the vertebra, is an area where injection of a fluid, typically bone cement, growth stimulant or other bone augmentation material, is a particularly common treatment, and such treatment is illustrative of many of the features on the subject matter described herein.

One common spinal condition that involves the injection of fluid as part of the treatment is referred to as vertebral compression fractures, or VCF. This refers to fracture of the vertebral body, which may occur in various ways, the most common of which is the result of osteoporosis, and is becoming more common as a result of the aging adult population. Of course, VCF can arise from other conditions, such spinal malignancies or impact accidents.

Referring to FIGS. 1-4 for background and general anatomical understanding, illustrated there are examples a human vertebral body that is healthy and one that that has been subject to a VCF. Specifically, FIG. 1 illustrates a portion of a healthy human spine or spinal column, generally designated as 100, free from injury. In contrast, FIG. 2 illustrates a vertebral column 128 having a VCF 134 in the middle vertebra 132.

The vertebral column 100 of FIG. 1 includes adjacent vertebrae 102, 102a and 102b and intervertebral disks 104, 104a, 104b and 104c separating the adjacent vertebrae. FIG. 3 illustrates in more detail a normal vertebra, as viewed looking down at the top of the vertebra, with a portion of the top or superior endplate 112 removed. FIG. 4 shows a normal vertebra, as viewed from the side.

Turning to FIG. 3, the vertebra, generally designated as 102, includes a vertebral body 106 that is roughly cylindrically, somewhat oval, and comprised of inner spongy cancellous bone 108 surrounded by the cortical rim 110, which is comprised of a thin layer of dense compact cortical bone. The body 106 of the vertebra is capped at the top by the superior endplate 112 and at the bottom by an inferior endplate 114, made of a cartilaginous layer (see FIG. 3). To the posterior (or rear) of the vertebral body 106 is the vertebral foramen 116, which contains the spinal cord (not shown). On either side of the vertebral foramen 116 are the bony pedicles 118, 118a, which lead to the spinous process 120. Other elements of the vertebra include the transverse process 122, the superior articular process 124 and the inferior articular process 126.

In the damaged vertebral column 128 of FIG. 2, vertebral body 130 of a vertebra 132 suffers from a compression fracture 134 on the front or anterior side of the vertebral body. As a result, the vertebral body 130 becomes typically wedge shaped, with consequent reduction in the height of both the vertebra 132 and vertebral column 128 on the anterior (or front) side. This reduction of height can also affect the normal curvature of the vertebral column 128, resulting in a more curved or hump-back appearance.

As mentioned earlier, vertebral compression fractures affect a large part of the population, and add significant cost to the health care system. As shown in FIG. 2, the vertebral compression fracture is a crushing or collapsing injury to one or more vertebrae. Vertebral compression fractures are generally, but not exclusively, associated with osteoporosis, metastasis, and/or trauma. Osteoporosis reduces bone density, thereby weakening bones and predisposing them to fracture. The osteoporosis-weakened vertebrae can collapse during normal activity and are also more vulnerable to injury from shock or other forces acting on the spine. In severe cases of osteoporosis, actions as simple as bending forward can be enough to cause a vertebral compression fracture. Vertebral compression fractures are the most common type of osteoporotic fractures according to the National Institute of Health.

One technique used to treat vertebral compression fractures is injection of a bone augmentation material directly into the fractured vertebral body. This procedure is commonly referred to as vertebroplasty. More particularly, vertebroplasty involves injecting fluid bone augmentation material (for example, bone cement, bone growth agent, allograph material or autograph material) into the collapsed vertebra to stabilize and strengthen the crushed vertebra. Other techniques for treating vertebral compression fractures employ the injection of bone cement into cavities formed within the cancellous bone of a vertebral body. Such cavities may be formed by removal of cancellous bone or by expansion of balloons within the vertebral body. Bone cement is also injected in or around implants that are inserted into the vertebral body to separate and support the vertebral endplates.

Injection of bone augmentation material into the vertebral body, however, sometimes carries with it the risk of “extravasation.” For example, in vertebroplasty and certain other procedures, bone augmentation material, and particularly bone cement, is introduced directly into the vertebral body with the physician preferably viewing the cement dispersion via fluoroscopy during the procedure. One drawback with this type of injection is the risk of undesired leakage or dispersion, called “extravasation,” of the bone augmentation material to undesired areas, such as into the vicinity of the spinal chord or nerves, which can be a major and serious complication. In fact, cement leakage has been reported in a wide range in vertebroplasty cases—ranging from 3% up to as much as 70% in VCF cases treated with vertebroplasty.

Accordingly, although the subject matter described herein is not limited to orthopedic or spinal treatments, it has particular benefits in the treatment of VCF, and is significant because there has been and continues to be a long felt need for devices and methods to control or limit flow fluids during injection into the body and in particular to control and/or limit the flow of bone augmentation material, such as bone cement and related fluids, injected into the vertebral body during vertebroplasty and certain other related VCF treatments.

### SUMMARY OF INVENTION

There are several aspects of the present subject matter which may be embodied separately or together in the devices, systems and methods described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a device is provided for controlled direction of fluid injection into the body. The device comprises an elongated, hollow cannula having a proximal end portion, a distal end portion, a shaft portion between the proximal and distal end portions, and a fluid flow lumen extending through the shaft portion between the proximal and distal end portions. The distal end portion is flexible and may preferably be pre-disposed or biased to assume a non-straight, e.g., curved, configuration having a radially inner side and a radially outer side. The radially inner side includes at least one aperture and preferably a plurality of apertures to direct fluid flowing from the lumen in a radially inward direction.

In another aspect, a device is provided for controlled direction of fluid injection into the body. The device comprises an elongated, substantially hollow cannula including proximal and distal sections. The injection assembly also includes an elongated guide member at least partially received within the cannula and including a core wire and a coil spring surrounding the core wire. The proximal section of the cannula has a greater stiffness than the distal section of the cannula. The device is particularly, but not exclusively, useful for injecting bone augmentation material into and through an implant located within a bone, such as a vertebral body.

### BRIEF DESCRIPTION OF THE DRAWINGS

FIG. 1 is a partial side view of a portion of normal healthy human vertebral column;

FIG. 2 is comparable to FIG. 1, but shows a vertebral compression fracture in one of the vertebral bodies;

FIG. 3 is a top view of a normal vertebral body with the superior endplate partially removed;

FIG. 4 is a side view of the vertebral body of FIG. 3;

FIG. 5 is a perspective view of an outer handle or first module with an introducer member according to an aspect of the present disclosure;

FIG. 6 is a perspective view of an injection or second module with an injection cannula according to an aspect of the present disclosure;

FIG. 7 is a perspective view of the injection module of FIG. 6 received within the outer handle module of FIG. 5 to form a fluid injection device, with the injection module in a retracted configuration with the distal end of the injection cannula located within the tubular member;

FIG. 8 is a perspective view of the fluid injection device or assembly of FIG. 7, with the injection module in a deployed configuration with the distal end of the injection cannula extending beyond the distal end of the tubular member;

FIG. 9 is a detail view of a distal end portions of the injection cannula and tubular member of FIG. 8;

FIG. 10 illustrates one embodiment of a fluid injection device employed in treating a fractured vertebral body;

FIG. 11 illustrates another embodiment of a fluid injection device in the treatment of a fractured vertebral body;

FIG. 12 is a top plan view of a first module with an injection cannula according to another embodiment of the present disclosure;

FIG. 13 is a top plan view of a second module with a guide member according to an aspect of the present disclosure;

FIG. 14 is a top plan view of the guide member of FIG. 12 received within the injection cannula of FIG. 13 to form a fluid injection device;

FIG. 15 is a detail view of the distal end of an alternative injection cannula according to an aspect of the present disclosure, with a portion thereof broken away for clarity;

FIG. 16 is a detail view of the distal end of another injection cannula according to an aspect of the present disclosure, with a portion thereof broken away for clarity;

FIG. 17 is a detail view of the distal end of yet another injection cannula according to an aspect of the present disclosure, with a portion thereof broken away for clarity;

FIG. 18 is a top plan view of an alternative guide member according to an aspect of the present disclosure;

FIG. 19 is a detail view of a distal portion of an injection assembly according to the present disclosure, with portions removed or transparent for clarity;

FIG. 20 is a top plan view of another alternative guide member according to an aspect of the present disclosure;

FIG. 21 is top plan view of another embodiment of an injection device according to the present disclosure;

FIG. 22 is a cross-sectional perspective view of a portion of the embodiment of FIG. 21;

FIG. 23 illustrates one embodiment of a fluid injection device employed in treating a fractured vertebral body, which may be employed with and/or as part of a vertebral body implant; and

FIG. 24 illustrates another embodiment of a fluid injection device in the treatment of a fractured vertebral body.

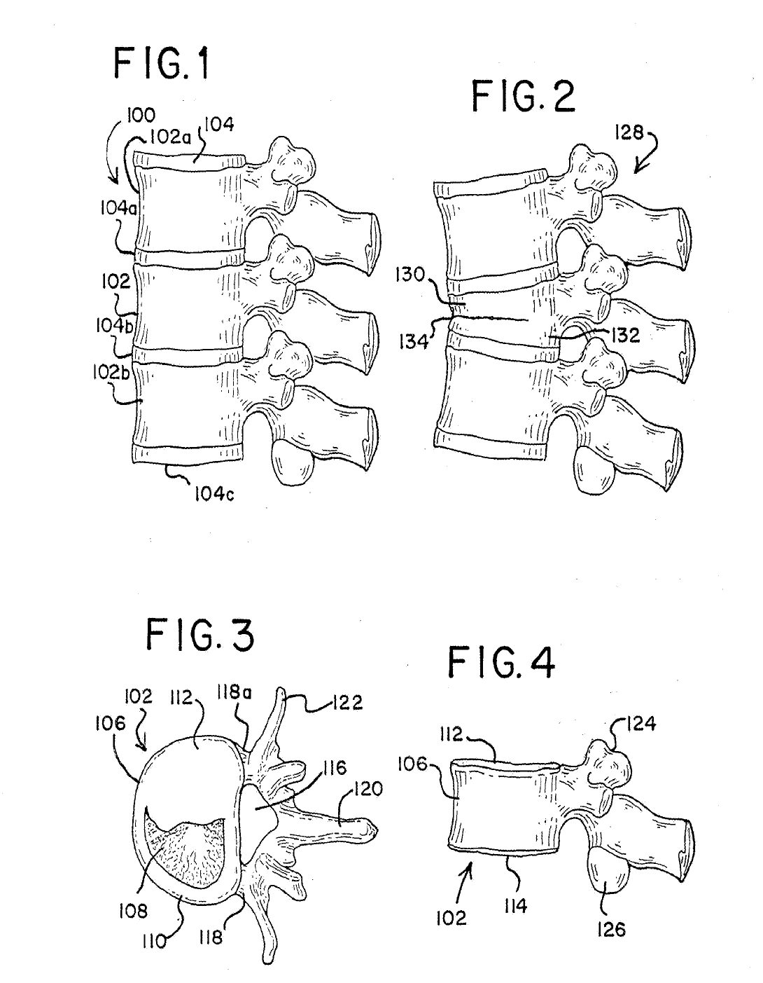

### DESCRIPTION OF EMBODIMENTS

The embodiments disclosed herein are for the purpose of facilitating a description of the present subject matter. They are exemplary only, and the subject matter herein may be embodied in various forms. Therefore, specific details disclosed herein are not to be interpreted as limiting the subject matter as defined in the accompanying claims.

Disclosed herein are devices or assemblies and methods for injecting fluid into a treatment site within a patient, especially bones including vertebrae for the treatment of compression fractures. The devices or assemblies for the directional control of fluid injected into the body include injection cannulas for delivery of fluid to a desired site such as a vertebral body. The injection cannulas are preferably elongated, hollow tubes or shafts defining a fluid flow lumen. At least a portion, preferably the distal end portion, of the injection cannula is sufficiently flexible to allow it to move from or between a generally straight constrained configuration and a non-straight configuration, such as the curved configuration. Such flexibility allows the injection cannula to adapt to a variety of shapes or configurations within a site such as a vertebral body. In addition, the flexibility of the injection cannula may also allow it to be received in a support device, such as a distraction device, implanted within a vertebral body. Examples of such distraction devices, include one such as illustrated in U.S. Pat. No. 7,666,226, which includes a passageway into which the injection cannula can be inserted, or others, such as illustrated in U.S. Pat. No. 6,595,998 or 5,755,797 for injection of cement around or in contact with a distraction structure, all of which patents are hereby incorporated by reference.

The injection cannula's flexibility may be achieved in any variety of ways, including but not limited to use of shape memory materials, a plurality of layers, varying thickness, incorporation of a tensioning member, curvature enhancing lateral slots or a combination of the above or other features. The distal end portion of the injection cannula may be curved by varying amounts, as desired. The curvature may range from a small amount of curvature to a full circle or coil and to a plurality of helical or spiral coils. More specifically, the curved distal end portion may vary from an arcuate shape (e.g., a 90° or 180° or 270° arc) or circular shape in a plane to a spiral or helical shape with a vertical dimension. As used herein, the term “spiral” refers to a coil-shaped structure with a non-uniform outer diameter, while the term “helical” refers to a coil-shaped structure with a substantially uniform outer diameter.

The injection cannulas of the present disclosure further preferably but not exclusively include at least one side aperture or port positioned in the distal end portion. The combination of the placement of the port(s) with the curved configuration of the distal end portion of the injection cannula permits directional delivery of fluid into a body. The port(s) are oriented to direct fluid to inject fluid in a direction generally transverse to the central longitudinal axis of the injection cannula and more specifically, when in the curved configuration in a radially inward direction (i.e., toward the center of curvature of the curved distal end portion).

The specific placement, number and configuration of the side port(s) may vary, depending on the desired ends to be achieved. The ports may be provided in any shape, such as but not limited to generally circular, elliptical, rectangular or square. If a plurality of ports are employed, the ports may be spaced apart along the axial length of the injection cannula and may be of the same size or vary in size along the injection cannula. It may be desirable for the flow rate of fluid through the injection cannula and exiting the side ports to be substantially equal as pressure changes throughout the cannula, in which case the size and shape of the side ports and the longitudinal separation between adjacent side ports may be customized to achieve that end. Various factors, including the nature of the fluid to be injected, the frictional coefficient of the injection cannula and the applied injection pressure affect the optimal placement, number and configuration of the side ports. Accordingly, these factors may be accounted for when designing the injection cannula. In general, for example, to achieve a substantially equal flow rate, the side ports are relatively small and spaced farther apart from each other at locations closer to the pressure source and larger and spaced closer together at locations farther from the pressure source.

It will be appreciated that, in contrast to an injection cannula having a curved distal end portion with an opening at its tip, an injection cannula having a curved distal end portion with more proximally positioned side port(s) or aperture(s) will generally result in more controlled (i.e., less diffusive) injection of fluid radially inwardly into a treatment site or core that is located radially inwardly of the curved distal end portion of the injection cannula. It should be appreciated that the presence of radially inwardly facing side port(s) does not preclude aperture(s) opening in other directions to further control the direction in which fluid is injected into the body. However, the radially outward side of the cannula may be solid or otherwise fluid imperious so as to block flow in a radially outward direction.

The injection fluid used in connection with the fluid delivery devices of the present disclosure will depend upon the procedure to be practiced with the device. Any variety of fluids may be used, including orthopedic cements and other bone augmentation materials, drugs, contrast agents, cell-based treatment materials such as stem cells, and other fluids, and for a variety of different diagnostic or therapeutic treatments or procedures. When treating a fractured bone, a bone growth stimulant and/or replacement/filler material or bone cement may be a suitable injection fluid. Such materials include but are not limited to a polymethyl methacrylate(PMMA) material or calcium phosphate, calcium sulphate, flowable hydroxyapatite (HA), any osteo-inductive or osteo-conductive material or bone morphogenic protein solution. Use of other fluids such as stem cell containing solution or other drugs or medication may be more appropriate for bone treatment and/or for other procedures. The fluid may also optionally include a radiopaque ingredient in order to facilitate imaging of the fluid delivery. It shall be appreciated that as used herein, “fluid” can refer to any substance or combination of substances that is capable of flowing under delivery conditions.

The fluid delivery devices and assemblies of the present disclosure may optionally include an introducer member that is configured to protect and/or restrain the injection cannula as it is introduced into the body, such as a vertebral body in particular. The introducer member preferably includes a tubular member that slidably receives the injection cannula between at least a retracted configuration and an extended or deployed configuration. The introducer member may further include an optional locking switch or mechanism which serves to maintain the injection cannula in fixed location or relation with respect to the introducer member. In some embodiments, the introducer member may further include a grip portion or handle for allowing the user to grasp and apply force to the tubular member.

Further, the devices and assemblies of the present disclosure may optionally include a guide member that is slidably received within an injection cannula. Like the injection cannula, at least a portion, preferably the distal end portion, of the guide member is sufficiently flexible to allow it to move from or between a generally straight, e.g., constrained, configuration and a non-straight configuration, such as a curved unconstrained configuration.

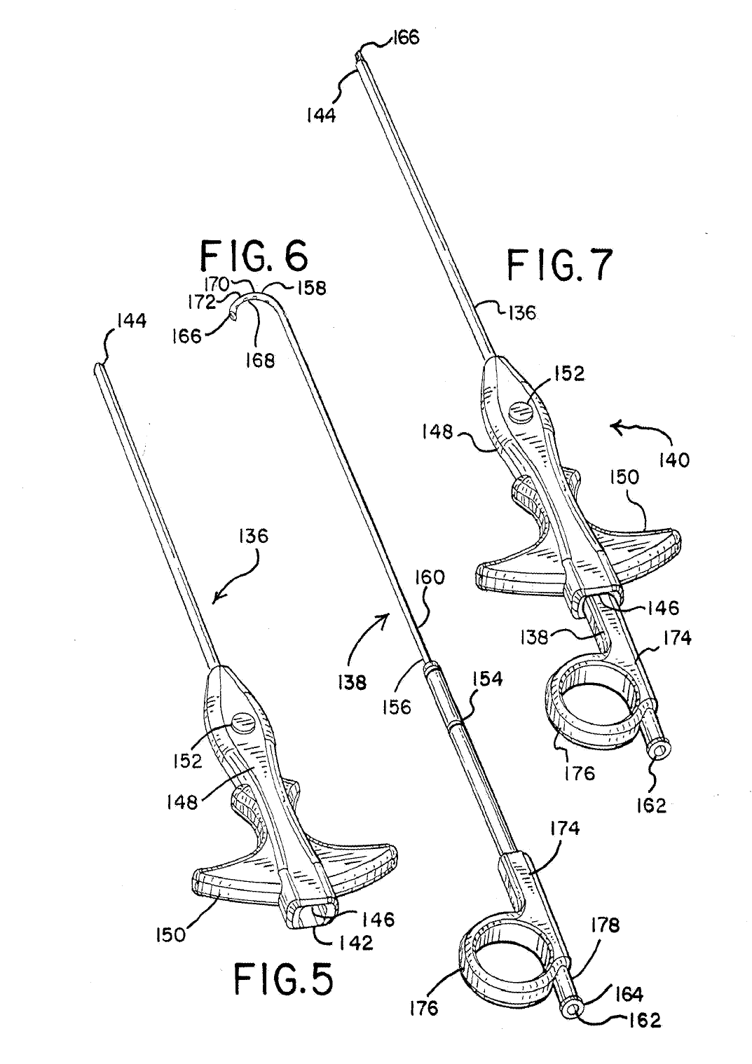

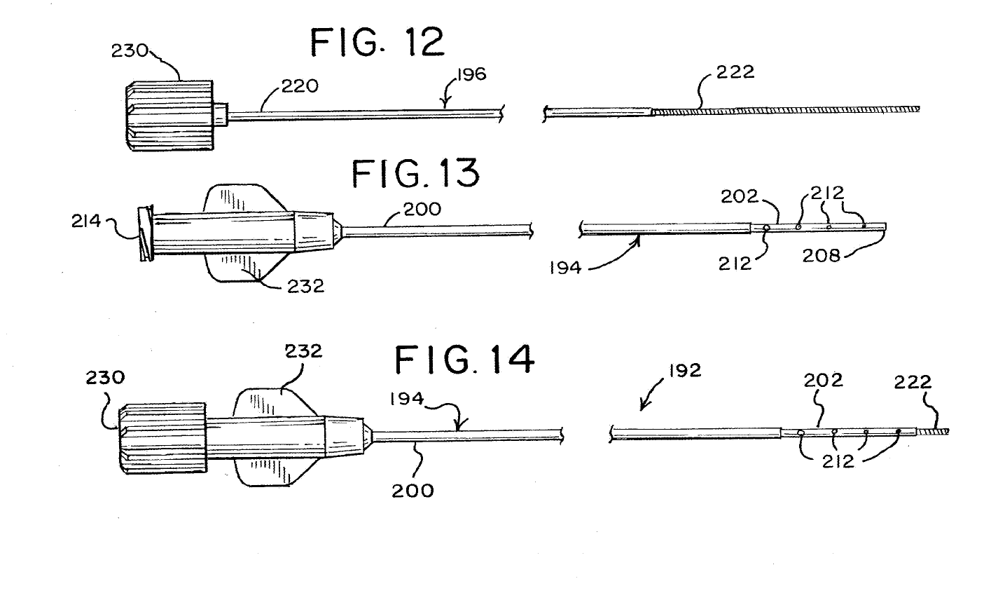

In one embodiment, the fluid delivery device may include an injection cannula and an introducer member (e.g. FIGS. 5-9). In another embodiment, the fluid delivery device may include an injection cannula and a guide member (e.g. FIGS. 12-14). It will also be appreciated that an injection device could include an injection cannula, introducer member and guide member. The fluid delivery devices or assemblies of the present disclosure may also include additional or fewer components without departing from the scope of the present disclosure. For example, a fluid delivery device of the present disclosure may include a supply of flowable injection fluid, such as a syringe or other type of pump connected to the injection cannula.

Turning to the drawings, one embodiment of a fluid delivery device or assembly 140 of the present disclosure is illustrated in FIGS. 5-9. The fluid injection device or assembly 140 includes a first component or module that includes an introducer member 136 (FIG. 5) and second component or module that includes an injection cannula 138 (FIG. 6) which is movable, e.g., slidable, axially within the tubular member 136 (FIGS. 7-9). The combination of the first and second modules or components is collectively referred to herein as an access or fluid delivery device or assembly 140.

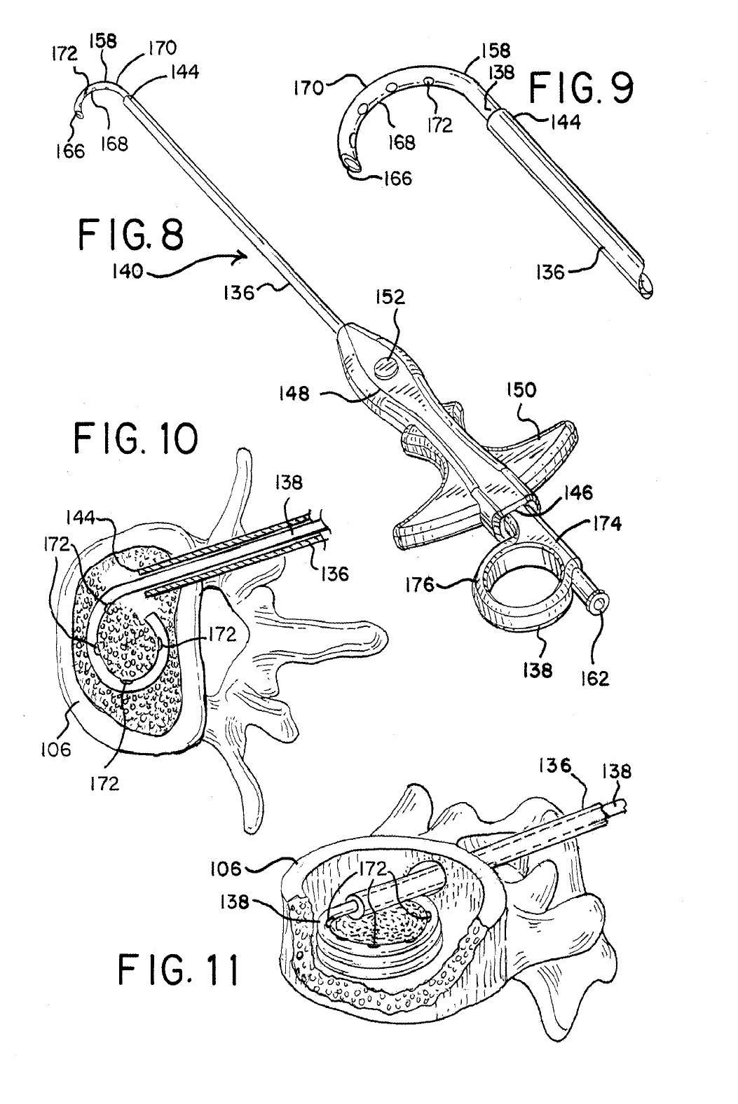

Turning to FIG. 5, the introducer member 136 has a lumen 146 that extends through an outer tubular member 137 between a proximal end opening 142 in handle 148 and a distal end opening 144. The lumen 146 is preferably sized and configured to slidably receive the injection cannula 138 (FIG. 6) between a retracted configuration (FIG. 7) and an extended or deployed configuration (FIG. 8), which will be described in greater detail herein.

For gripping and applying force to the outer cannula 137, the proximal portion of the introducer member 136 may include the grip portion or handle 148 configured to be grasped or otherwise manipulated by hand and suited to withstand substantial axial force as the fluid delivery device 140 is moved through body tissue. It may be advantageous for the grip portion 148 to be ergonomically designed, for example with laterally extending finger grips 150 or the like for improved handling and control of the fluid delivery device 140 during use. Other shapes for the grip portion 148, other than the illustrated generally T-shaped embodiment, may also be employed without departing from the scope of the present disclosure.

The distal portion of the outer tubular member 137 extending distally beyond the grip portion 148 may be comprised of any of a number of substantially rigid materials such as, but not limited to, stainless steel, rigid high strength plastic or any other high strength material. It may be advantageous for the distal portion to be comprised of a radiopaque material, if not made of metal, or to include one or more radiopaque markers to allow for visualization of the distal portion under x-ray or other imaging while in the body. As for the grip portion 148, it may also be made of a rigid material (to withstand axial force as the user positions or moves the outer tubular member 136 through body tissue), though some degree of pliability or softness may also be advantageous (for improved comfort). Suitable materials for the grip portion 148 include, but are not limited to, rigid plastic. The manner in which the grip portion 148 is secured to the outer tubular member 137 is essentially an engineering choice and will depend on the material composition of the grip portion 148 and the distal portion, but may include, for example, the use of adhesives, a friction fit, keying and/or overmolding.

The introducer member 136 may further include an optional locking switch or mechanism 152 which serves to maintain the injection cannula 138 in fixed location or relation with respect to the outer tubular member 137. The locking mechanism 152 may be variously configured, but in the illustrated embodiment, it is a button or switch which is movable radially toward and away from the central axis of the lumen 146. FIG. 7 shows the locking mechanism 152 in a radially outward or locked position and FIG. 8 shows the locking mechanism 152 in a radially inward or unlocked position. The locking mechanism 152 may be biased to the radially outward position by a spring or other biasing member (not shown). In the radially outward position, a portion of the locking mechanism 152 engages the injection cannula 138 to hold the injection cannula 138 in place axially with respect to the outer tubular member 137. It may be advantageous to lock the injection cannula 138 in place in the retracted configuration of FIG. 7, but the injection cannula 138 may be lockable in place in any variety of other positions or multiple positions, employing any suitable locking structure.

Turning to FIG. 6, the injection cannula 138 may include a mating feature, such as the annular groove 154, designed to mate, correspond or cooperate with the locking mechanism 152 of the introducer member 136 when the locking mechanism 152 is in the locked or radially outward position. When the locking mechanism 152 is pressed down (i.e., moved into the lumen 146 of introducer member 136 or towards its central axis), the locking mechanism 152 disengages from the mating feature 154 of the injection cannula 138, thereby releasing the injection cannula 138 and allowing it to move with respect to the introducer member 136, such as to the extended configuration of FIG. 8.

The injection cannula 138 is preferably elongated and hollow, with a proximal end portion 156, a distal end portion 158, and a shaft portion 160 therebetween. A fluid flow lumen 162 extends though the shaft portion 160 between the proximal and distal end portions 156 and 158 and the proximal end of the fluid flow lumen 162 optionally terminates in a handle portion 174, which includes an opening 164 for receiving injection fluid therein. It will be appreciated that the fluid flow lumen could terminate at other positions along the shaft 160 and not depart from the spirit of the present disclosure.

In one embodiment, the distal-most end or tip 166 of the injection cannula 138 is closed or otherwise sealed and may optionally be sharpened or pointed. As shown in FIG. 7, in the retracted configuration the tip 166 of the injection cannula 138 extends slightly beyond the distal end opening 144 of the introducer member 136, meaning that tip 166 will be the first part of the fluid delivery device 140 that engages body tissue as the device or assembly 140 is advanced toward a treatment site. Accordingly, providing a pointed tip 166 may make it easier to push, advance or force the fluid delivery device 140 through body tissue.

As mentioned above, the distal end portion 158 of the injection cannula 138 is also sufficiently flexible to allow it to move from or between the generally straight constrained configuration of FIG. 7 to a non-straight configuration, such as the curved configuration of FIGS. 6 and 8-9 (which will be described in greater detail herein). To achieve a distal end portion 158 with the desired combination of rigidity and flexibility, it may be advantageous for at least the distal end portion 158 to be comprised of a shape memory material, such as Nitinol or other nickel titanium alloy. A distal end portion so configured may be manufactured, such as by heat setting, cold forming or other known techniques so as to be predisposed to assume a curved configuration when the injection cannula 138 is advanced beyond the distal end opening 144 of the outer tubular member 136 and is no longer constrained within the lumen 146 of the introducer member 136 (FIGS. 8 and 9).

Another construction for moving the distal end portion from a straight to a non-straight configuration may be the employment of tensioning member. For example, a tensioning member, such as a pull wire (not shown), may extend through or along injection cannula 138. The pull wire may be attached at a distal end to the distal end portion 158 of injection cannula 138 and extend to a user-accessible proximal end portion that allows tensioning of the pull wire to cause the distal end portion to bend, with the degree or extent of curvature being selectable depending upon the amount of tension exerted. The proximal end of the pull wire may be attached to any suitable mechanism, such as a thumb-knob, rotary actuator or other for applying or releasing tension on the guidewire.

The curved configuration of the injection cannula 138 of FIGS. 6 and 8-9 is merely exemplary. As mentioned above, it should be understood that the exact curvature of the distal end portion 158 may vary. The curvature of the distal end portion 158 illustrated in FIG. 9 is 180°. FIG. 9 illustrates the assembly with a distal end portion 158 of the injection cannula 138 advanced from the distal end of the introducer member 136, FIG. 10 illustrates a similar position during treatment of vertebral body and FIG. 11 illustrates an injection cannula 138 in which the distal end portion 158 is advanced within a vertebral body and assumes a predisposed helical configuration with vertically arranged windings or loops (of any desired number) that encircle a region or core of cancellous bone. During winding, it is noted that the cancellous bone is essentially undisturbed.

The curved configuration of the distal end portion 158 of the injection cannula 138 permits directional delivery of injection material. More particularly, the distal end portion 158 has a radially inner bend or side 168 and a radially outer bend or side 170 when in the curved configuration (see FIG. 9). As used herein, the phrase “radially inner” refers to a direction facing generally toward the center of curvature of the curved distal end portion 158, while the phrase “radially outer” refers to a direction facing generally away from the center of curvature of the curved distal end portion 158. The distal end portion 158 includes at least one aperture or port 172 positioned proximally of the distal-most end or tip 166 and oriented to direct injection fluid from the lumen 162 in a radially inward direction (i.e., toward the center of curvature of the curved distal end portion 158). In the illustrated embodiment, the radially inner side 168 includes one or more ports 172 located along the axial length of injection cannula. The radially outer side 170 of the distal end portion 158 may be solid so as to substantially block or prevent fluid flow in a radially outward direction, for example, by being free of any fluid flow apertures or ports. However, the shape, size, positioning and longitudinal spacing of the ports 172 are not limited to a specific configuration illustrated, but may vary depending upon the particular application or use.

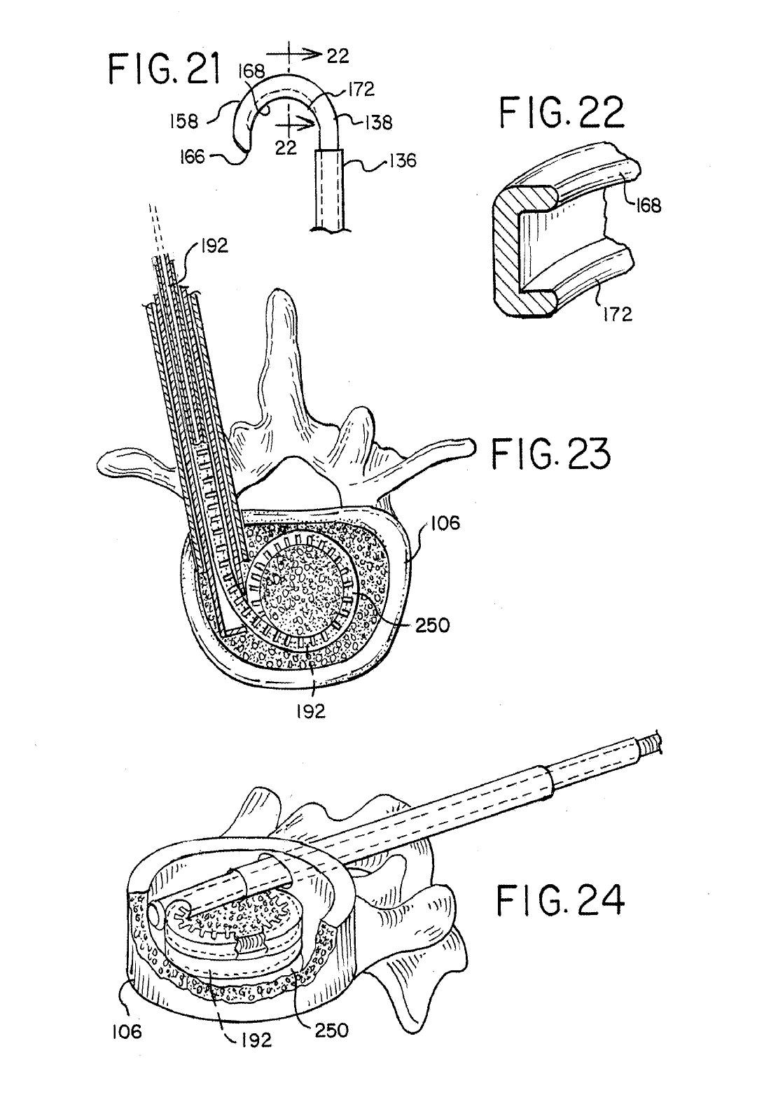

Another embodiment of an injection cannula of the present disclosure is illustrated in FIGS. 21 and 22. In this embodiment, injection cannula has a generally rectangular cross section and includes one elongated side port positioned at least in the distal end portion 158. As illustrated, the side port is a rectangular opening defined substantially in the radially inner wall of the cannula resulting in an injection cannula having a generally “c-shaped” cross section along the portion where the port is located. However, it will be appreciated that the side port and cannula could have other configurations without departing from the spirit of the present disclosure. For example, the side port could be formed as a slot in the radially inner wall or the cannula may have a generally circular cross section. While the injection cannula is illustrated with a pointed and closed distal-most end or tip 166, in alternative embodiments, the port could continue through the distal-most end or the tip could be made blunt. However, as configured, one skilled in the art would understand that the injection cannula may also be used as a channel creation device in order to create a channel in a body such as a vertebral body and more specifically the cancellous bone therein.

Returning to FIGS. 5-11, it may be advantageous for the distal end portion 158 to curve about a known axis to make the injection procedure more predictable/controllable. For example, the distal end portion 158 may be configured to curve about an axis extending at an angle, including up to about 90°, to the plane of the longitudinal axis of the distal end portion or to the shaft portion 160. As the distal end portion 158 will be positioned at a treatment site within the body when it is moved into its curved configuration, it may be advantageous for the orientation of the curved configuration to be ascertainable from outside of the body. In one embodiment, the proximal end portion 156 of the injection cannula 138 further includes a handle 174 with a plane indicating structure 176, such as a laterally extending thumb loop for gripping the handle 174. The thumb loop 176 defines a plane and, in one embodiment, the distal end portion 158 of the injection cannula 138 is predisposed to curve substantially within that plane, in the direction of the thumb loop 176. If the distal end portion 158 is configured to assume a helical shape, it may start by curving within the plane before coiling vertically about an axis perpendicular to the plane (and to the shaft portion 160 of the injection cannula 138). By such a configuration, a user will know the predisposed orientation of the curved distal end portion 158 just by visually and/or tactilely referring to the position of the laterally extending structure 176, which remains outside of the body during use.

The handle 174 may further include a fluid connector 178, such as a luer fitting, which allows the injection cannula 138 to be removably connected to a fluid source, as described above. Alternatively, the fluid source may be preconnected to the handle, requiring only the opening of a valve, frangible connector or other flow control device. The handle 174 may be comprised of a different material than the injection cannula 138. For example, the handle 174 may be made of the same material as the grip portion 148 of the first module.

In a method of using a fluid delivery device 140 according to the present disclosure to inject fluid into a fractured vertebral body, the injection cannula 138 is initially retracted so as to keep the distal end portion 158 restrained within the straight introducer member 136 and the pointed distal-most end or tip 166 protruding from the distal end opening 144 of the introducer member 136. If provided, the locking mechanism 152 of the introducer member 136 may be engaged with the mating feature 154 of the injection cannula 138 to prevent the injection cannula 138 from moving away from the engaged or locked position of FIG. 7.

With the fluid delivery device 140 so configured, the user (physician) inserts the pointed distal-most end or tip 166 through a small incision in a patient's back and advances the end 166 through, for example, the pedicle of the patient's fractured vertebral body and into the desired treatment position in the vertebral body. Typically, the user, directly or indirectly, grasps the handle or grip portion 148 of the outer tubular member 136 while moving or otherwise positioning the device 140 into the vertebral body. The distal end of the fluid delivery device 140 is placed substantially vertically central and slightly anterior of central A-P (anterior to posterior). It may be advantageous for the trajectory of the device 140 to be slightly away from medial, depending on which pedicle the device has been introduced through. As discussed above, when advanced, the distal end portion 158 of the injection cannula 138 will curve in the direction and plane of the thumb loop 176, so the device 140 should be positioned and held accordingly such that there is sufficient room to accommodate the curved portion. Alternatively, position of the distal end portion 158 may be viewed real time via x-ray or other position-indicating means provided. Accordingly, the illustrated device 140 should be positioned to the right of medial such that the center of the deployed arc will preferably be laterally centered.

Once the device 140 is positioned at the target site, the locking mechanism 152 can be disengaged to uncouple the injection cannula 138 and the outer tubular member 136. Using fluoroscopic imaging to ensure safe advancement of the distal end portion 158, the handle 174 of the injection cannula 138 can be advanced slowly in the distal direction (while holding the introducer member 136 stationary) to the any desired deployed configuration (e.g. see FIG. 8, 9, 10 or 11). As noted earlier, the distal end portion 158 of the injection cannula 138 may be curved by varying amounts, as desired, ranging from a small amount of curvature to a full circle or coil and to a plurality of helical or spiral coils. If the device is employing a tensioning member to cause bending of the distal end portion, the amount of curvature can be varied by varying the amount of tension applied to a pullwire (not shown). The device that applies the tension can be of any suitable design such as a rotating knob actuator, thumb slide, and other mechanisms. The injection cannula could be advance in a straight configuration through an outer cannula of the introducer member, and tension applied as or after the distal end portion exits the outer cannula. Under this configuration, the injection cannula 138 does not need to be locked in the deployed configuration because the curved distal end portion 158 has the resistance of the body tissue and acts as an anchor. Alternatively, the locking mechanism 152 may be actuated to lock the injection cannula 138 and introducer member 136 together in the deployed configuration. In one embodiment, a portion of the handle 174 (e.g., the thumb loop 176) engages the proximal end or the grip portion 148 of the outer tubular member 136 when the distal end portion 158 has been fully deployed. Such a configuration gives a tactile and visual indication of full deployment of the curved distal end portion while also preventing over-extension of the injection cannula 138.

Once the distal end portion has been deployed as desired, a syringe or other fluid source can be attached to the injection cannula 138 at fluid connector 178 and the fluid injection process may be initiated. Alternatively (or additionally), the lumen 162 of the cannula 138 can be preloaded with a predetermined amount of injection fluid. During fluid injection, the fluid will be advanced through injection cannula 138, out of the ports 172 of the distal end portion 158, and into the vertebral body. Fluoroscopic imaging may be used to verify that the desired delivery and dispersion of fluid is achieved (provided that the injection fluid is radiopaque). As a result of the configuration of the curved distal end portion 158 and the positioning of the port(s) 172, the fluid dispersion is very well controlled and tends to be contained within a central portion of the vertebral body, for example within largely undisturbed cancellous bone, thus tending to provide better control so that the fluid will not migrate to an undesirable region of the patient (e.g., into the vicinity of the spinal cord).

Upon completion of the fluid injection, the curved distal end portion 158 is retracted by moving handle 174 and thus the entire injection cannula 138 proximally with respect to the outer tubular member 136. Once fully retracted, the locking mechanism 152 may be re-engaged and the device 140 may be carefully removed from the patient's back. In one embodiment, the injection cannula 138 can be retracted further into the outer tubular member 136 (i.e., more proximally than the initial position of FIG. 7) and locked in a third, most proximal position for extra safety. Once the device 140 is removed from the patient, the small incision through the skin may be sutured or otherwise cleaned and closed. Although only shown in use by itself, the fluid delivery devices may be employed in combination with other structures or devices, such as support structures which have been implanted into the vertebral body 106. More specifically, the injection cannula could be introduced within an interior lumen of a distraction device.

Another embodiment of a fluid delivery device 192 according to the present disclosure is illustrated in FIGS. 12-14. The fluid delivery device 192 comprises a first component or injection cannula 194 and a second component or guide member 196. FIGS. 12 and 13 illustrates the injection cannula 194 and guide member 196 separate from each other, while FIG. 14 shows them assembled, with the guide member 196 slidably received in a central lumen or fluid passageway of the injection cannula 194. As mentioned above, the fluid delivery device 192 may be used in conjunction with other components, such as a supply of fluid.

The injection cannula 194 is preferably elongated and substantially hollow with an inner lumen configured to selectively receive guide member 196 during transport and/or insertion to inject fluid into the vertebral body when the guide member 196 has been removed. The proximal and distal ends of the injection cannula 194 are open, which allows for fluid flow from a fluid source associated with the proximal end portion or end 200 of the injection cannula 194, through the lumen of the injection cannula 194, and out the distal end portion or end 202 of the injection cannula 194. As for the outer diameter of the injection cannula 194, it is preferably sized so as to allow the injection cannula 194 to be received within a vertebral body and preferably sized to be slid through a support structure, such as a distraction device, implanted therein.

Rather than being rigid, the injection cannula 194 is preferably at least partially flexible, allowing it to be advanced into a vertebral body. The injection cannula is preferably sufficiently flexible to allow it to move from or between a generally straight constrained configuration and a non-straight configuration, such as the curved configuration. More specifically, the injection cannula 194 is sufficiently flexible to allow it to pass through a support structure such as an implant positioned within the vertebral body in a curved, arced or coiled configuration. In one embodiment the distal end portion 202 of the injection cannula 194 is substantially flexible, and the proximal end portion 200 is relatively less flexible or even substantially inflexible. The stiffer proximal end portion 200 gives the injection cannula 194 a degree of axial columnar strength or stiffness, which helps when pushing the injection cannula 194 through an implanted distraction device within the vertebral body by preventing, kinking, “snaking,” or buckling. The more flexible distal end portion 202 allows the distal end of the injection cannula 194 to track along a curved or coiled support structure, such as an implant, within a vertebral body.

The flexible distal end portion 202 allows the injection cannula 194 to inject fluid into the vertebral body and more specifically, different regions of a support structure and, if the distal end portion 202 is sufficiently elongated (i.e., at least the same length as the distraction device 250), can allow for fluid injection into any location or plurality of locations within the support structure. Depending on the nature of the support structure and the procedure, the relative lengths of the proximal and distal end portions 200 and 202 may vary. In an exemplary embodiment, the proximal end portion 200 is significantly longer than the distal end portion 202, with the proximal end portion 200 being several inches long (e.g., approximately 7 inches long) and the distal end portion 202 being a fraction of an inch (e.g., approximately 0.75″ long). It will be appreciated that injection cannulas having proximal and distal end portions 200 and 202 of other lengths may also be employed without departing from the scope of the present disclosure.

As mentioned above, the varying flexibility of the injection cannula 194 along its length may be achieved in a number of ways. For example, in the embodiment illustrated in FIG. 12, the injection cannula is comprised of two components, with a proximal component 200 being comprised of a relatively rigid or less flexible tubular material, such as, but not limited to, a metal (e.g. stainless steel) and a distal component 202 being comprised of a more flexible tubular material, such as, but not limited to, a non-metallic, polymeric material (e.g. high density polyethylene, PEBAX® available from Arkema, Inc., fluoropolymer, and/or nylon). The components may be joined by adhesion, welding, thermal bonding, or other suitable joining technique.

In another embodiment, the injection cannula is comprised of a plurality of layers which can be customized to vary the flexibility of the injection cannula in different end portions. The distal end of such an injection cannula 194 is illustrated in FIG. 15. In this embodiment, at least the distal end portion 202 of the injection cannula 194 is comprised of an inner layer 204 and an outer layer 206, with the inner layer 204 having a greater stiffness or rigidity than the outer layer 206. The stiffness of the inner layer 204 prevents the injection cannula 194 from “snaking” or buckling as the injection cannula 194 is moved into the vertebral body and more specifically through a support structure implanted therein. The softer outer layer 206 prevents the injection cannula 194 from catching or snagging on the support structure as the injection cannula 194 is moved therethrough, thereby protecting both from damage and wear. To provide additional protection, the outer layer 206 of the injection cannula 194 may have an atraumatic or formed (e.g., curved or beveled) distal end 208, as shown in FIG. 15.

The inner and outer layers 204 and 206 of the injection cannula 194 may be made of a flexible material, such as a polymeric material. The inner and outer layers 204 and 206 may be joined to each other by any of a number of means, which may vary according to the nature of the material composition of each. In one embodiment, the inner and outer layers 204 and 206 are joined by thermal processing (e.g., adding the outer layer 206 to the inner layer 204 as a shrink tube, re-flow melting the outer layer 206, molding the outer layer 206 onto the inner layer 204, or employing a co-extrusion process).

In an alternative embodiment illustrated in FIG. 16, the injection cannula may include a third or reinforcing layer 210 that provides a reinforcement or stiffening affect. It will be appreciated that such reinforcing layer may be associated with only the proximal end portion 200 of the injection cannula 194 to give it a greater stiffness than the distal end portion 202. Alternatively, the third layer 210 may be present in the distal end portion 202, but present in a greater amount in the proximal end portion 200 to give the proximal end portion 200 a greater stiffness.

The reinforcing layer 210 may be variously configured without departing from the scope of the present disclosure, but in one embodiment, it is comprised of a material with a stiffness greater than that of the inner and outer layers 204 and 206 (such as, but not limited to, KEVLAR® or stainless steel). The reinforcing material may be provided in a layer which is a wound, braided, or woven. In an alternative embodiment, the reinforcing layer 210 may be sandwiched between the inner and outer layers 204 and 206, with the joinder of the inner and outer layers 204 and 206 to each other effectively securing the reinforcing layer 210 as well. Other means for securing the reinforcing layer 210 within the injection cannula 194 may also be employed without departing from the scope of the present disclosure. Additional layers could also be provided if desired.

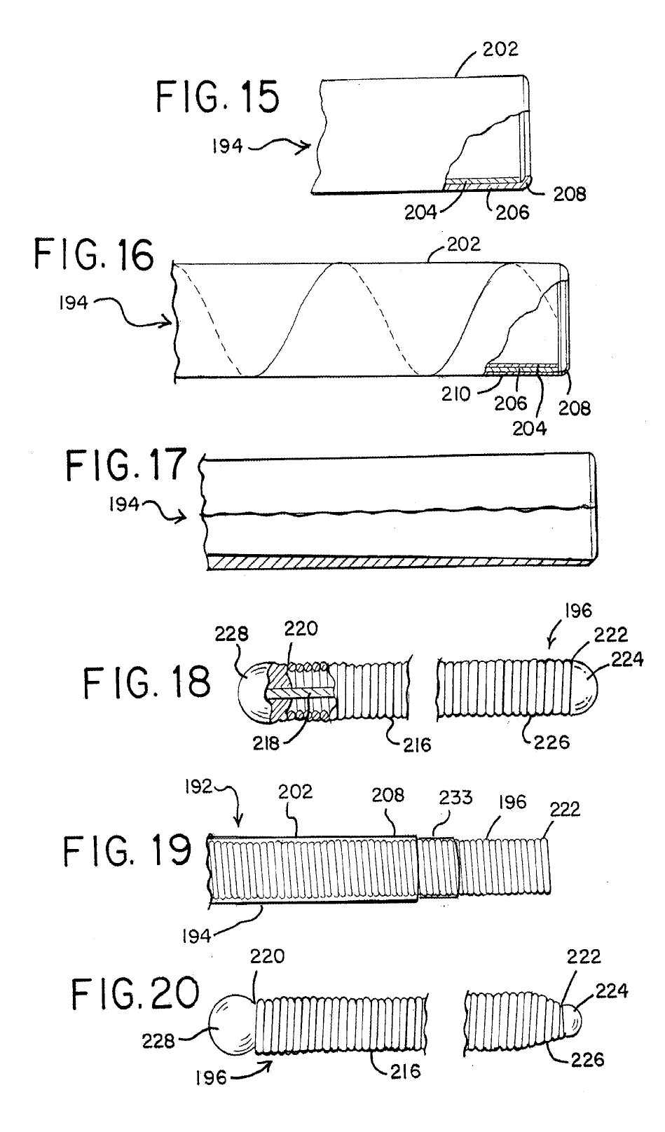

In yet another embodiment illustrated in FIG. 17, the injection cannula 194 is comprised of a single component with a varying thickness. The proximal end portion of the injection cannula may be thicker than the distal end portion, resulting in a distal end portion that is more flexible than the proximal end portion.

Regardless of its configuration to obtain the desired flexibility, the injection cannula 194 may be provided with a plurality of apertures or ports 212 (see FIGS. 13 and 14) to provided for the delivery of the injection fluid. In the illustrated embodiment, the injection ports 212 are longitudinally spaced along a side of the injection cannula 194 such that when the injection cannula 194 has been at least partially inserted into a vertebral body and more specifically into a support structure therein, the side ports 212 promote the injection of fluid into a resident volume. If desired, an opening in the very distal end or tip of the injection cannula may also be included. It may be preferred for the side ports 212 to be positioned in only the portion of the injection cannula 194 that will be positioned within the curved or coiled portion of the support structure, e.g. the distraction device. In general, adding side ports to any portion of the injection cannula 194 will decrease the stiffness of that portion, meaning that it may be preferable to only incorporate the side ports 212 in the distal end portion 202 of the injection cannula 194 (which is advantageously flexible) and not in the proximal end portion 200 (which is advantageously less flexible). As noted above, alternative embodiments may include any number of side ports which may be positioned anywhere along the length of the injection cannula and it should be understood that the embodiment shown if FIGS. 13 and 14 is merely one example of a possible injection cannula 194.

Another goal may be to deliver specific, varying amounts of fluid to different sections of the vertebral body (e.g., if it is believed that more fluid is needed at an upper section of the resident volume as opposed to a lower section), in which case at least one side port may be larger than another side port to achieve that end, as generally illustrated in the embodiment of FIG. 13.

Turning to the second component (FIG. 12), the elongated guide member 196 is substantially cylindrical and configured to be slidably received within the lumen of the injection cannula 194. Similar to the injection cannula 194, it may be advantageous for the guide member 196 to have a distal end portion 222 which is substantially more flexible and a proximal end portion 220 which is less flexible or even substantially inflexible. The stiffer proximal end portion 220 gives the guide member 196 (and, hence, the fluid delivery device 192) a degree of column strength or stiffness, which helps when pushing the fluid delivery device 192 to and into the vertebral body and more specifically into a support structure therein. The guide member's more flexible distal end portion 220 allows the distal end of the fluid delivery device 192 to track along a curved or coiled support structure. Depending on the nature of the support structure and the procedure, the relative lengths of the proximal and distal end portions 220 and 222 may vary. In an exemplary embodiment, the proximal end portion 220 is significantly longer than the distal end portion 222, with the proximal end portion 220 being several inches long (e.g., approximately 7 inches long) and the distal end portion 202 being substantially shorter such as 20% of the length of the proximal end portion more or less (e.g., approximately 1.5 inches long). Guide members having proximal and distal end portions 220 and 222 of other lengths may also be employed without departing from the scope of the present disclosure.

Like the injection cannulas of the present disclosure, the varying flexibility or stiffness of the guide member 196 along its length may be achieved in a number of ways. For example, in one embodiment the guide member 196 is comprised of two components, with a proximal component being comprised of a relatively rigid or less flexible hollow or solid generally cylindrical material (such as, but not limited to, a metallic material, such as stainless steel) and a distal component being comprised of a more flexible hollow or solid generally cylindrical material (such as, but not limited to, a polymeric material, such as high density polyethylene, fluoropolymer, and/or nylon). The components may be joined by crimping, adhesive bonding, thermal bonding, or other suitable attachment mechanism.

In another embodiment, the guide member is provided which is relatively rigid in an axial direction (to allow the fluid delivery device 192 to be advanced through a support structure), but flexible in a radial direction (to allow the fluid delivery device 192 to track along the curved lumen of a support structure).

FIG. 18 is an illustrated example of such an embodiment, the body of the guide member 196 may be comprised of a flexible coil spring 216 attached to an inner core wire 218. The core wire 218 is an elongated shaft or element extending from the proximal end portion or end 220 of the guide member 196 and provides resistance to buckling as the fluid delivery device 192 is being deployed. The core wire 218 of FIG. 18 is shown with an outer diameter which is much smaller than the inner diameter of the coil spring 216, but may be larger than shown, with an outer diameter which may be large enough to approximate the inner diameter of the coil spring 216. If a core wire having a relatively large outer diameter is provided, it may be advantageous for the core wire to be tapered, for example, with a large diameter at its proximal end (for enhanced stiffness) and a smaller diameter at its distal end (for enhanced flexibility).

Other modifications may also be made to the configuration of the core wire 218 without departing from the scope of the present disclosure. For example, the core wire 218 may either extend along the entire length of the guide member 196 or, instead, extend along only a portion of the length of the guide member 196. If the core wire 218 extends along the entire length of the guide member 196, it may be advantageous for the core wire 218 to be tapered, having a smaller cross-end portional area at its distal end than at its proximal end to make the distal end (particularly the portion which enters into the lumen of a distraction device) relatively more flexible.

Alternatively, if the core wire 218 extends along only a portion of the length of the guide member 196, it may be advantageous for the core wire 218 to extend from the proximal end 220 of the guide member 196, but stop short of the distal portion of the guide member 196 which is required to bend to track along the lumen of a coiled distraction device.

The coil spring 216 preferably surrounds the core wire 218 and extends from the proximal end 220 of the guide member 196 to the distal end portion or end 222. The coil spring 216 gives the guide member 196 a degree of lateral flexibility, particularly the distal end portion of the guide member 196 if the coil spring 216 extends beyond the distal tip of the core wire 218. It may be advantageous for the coil spring 216 to be tightly coiled (i.e., with little or no separation between adjacent coils) so as to be substantially incompressible and at least semi-rigid when an axial force is applied to the guide member 196 (e.g., when the fluid delivery device 192 is being advanced to a treatment site).

As shown in FIGS. 14 and 19, when the guide member 196 is received within the injection cannula 194, the distal end 222 of the guide member 196 may extend beyond the distal end 208 of the injection cannula 194. If the fluid delivery device/system 192 is so configured, then the distal end 222 of the guide member 196 will be the first part of the fluid delivery device 192 which enters into the vertebral body and more specifically the support structure therein as the fluid delivery device 192 is being deployed to a treatment site. Accordingly, it may be advantageous for the distal end 222 of the guide member 196 to be atraumatic or otherwise formed to prevent the guide member 196 from catching or snagging on the slots 172 of the distraction device 166 as the fluid delivery device 192 is moved through the lumen 173 thereof. The distal tip of the coil spring 216 may be secured (e.g., by welding or any other means) to a semi-spherical or otherwise atraumatic cap 224. If the core wire 218 extends the length of the guide member 196, it may also be secured to the cap 224 and/or the distal end 226 of the coil spring 216. Additionally, the distal end 226′ of the coil spring 216 may also be tapered to a decreased outer diameter (FIG. 20) to further aid in introducing the fluid delivery device 192 into a support structure within the vertebral body.

The proximal tip of the coil spring 216 is shown in FIGS. 18 and 20 as also being secured to a second atraumatic cap 228, although that portion of the guide member 196 will not be advanced into the distraction device, so any cap member may be employed. For example, the proximal ends of the core wire 218 and coil spring 216 may include mating connectors allowing them to be temporarily secured to each other. In one embodiment, which is shown in FIGS. 12 and 14, the proximal end 220 of the guide member 196 is provided with a luer cap 230, which can be releasably secured (e.g., by mating threads) to a luer hub 232 associated with the proximal end 214 of the injection cannula 194. This or any other suitable means for temporarily securing the injection cannula 194 and guide member 196 to each other may be advantageous to ensure that they move simultaneously as the fluid delivery device 192 is advanced toward the treatment site.

The core wire 218 and coil spring 216 may be made of any of a variety of suitable materials including, but not limited to, metallic materials. It may be advantageous for the core wire 218 and coil spring 216 to be made of the same material (or at least compatible materials) if they are to be secured directly to each other. It may be further advantageous for one or both of them to be comprised of a radiopaque material to allow for improved visualization of the guide member 196 while it is in situ. In particular, the coil spring 216 and core wire 218 may be comprised of stainless steel, high tensile steel wire, or nitinol, although other materials may also be used without departing from the scope of the present disclosure.

The coil spring 216 may be partially or completely surrounded by a thin sheath or outer layer 233 (e.g. a shrink tube), which can be seen in FIG. 19. Such a sheath 233 may have lower friction than the coil spring 216, which allows for a close fit to the inner diameter of the injection cannula 194 while also preventing the coil spring 216 from damaging or becoming lodged within the injection cannula 194. A close fit between the guide member 196 and the injection cannula 194 may be advantageous to improve the trackability of the injection cannula 194 as it is being deployed. Additionally, the sheath 233, if provided, increases the stiffness of the guide member 196 and helps to prevent unwinding or stretching of the coil spring 216 during removal of the guide member 196 from the injection cannula 194 (i.e., when the coil spring 216 is placed in tension). Suitable materials for a sheath 233 include, but are not limited to, PEBAX®, polyolefin, or fluoropolymer.

In a method of using a fluid delivery device 192 according to the present disclosure to inject fluid into a distraction device 250 implanted within a fractured vertebral body, the fluid delivery device 192 can be advanced into the lumen of the distraction device 250 (FIG. 23). As the fluid delivery device 192 enters the curved or coiled distraction device 250, the devices flexible distal end tracks along the curves until it reaches the desired location. Typically, the optimal location for the distal end of the fluid delivery device 192 depends on the location of the side ports 212 of the injection cannula 194, although other considerations also affect the degree to which the fluid delivery device 192 is inserted into the distraction device.

With the fluid delivery device 192 in the desired location, ideally with the side ports 212 of the injection cannula 194 facing radially inwardly within the distraction device 250 (i.e., toward the center of the resident volume), the guide member 196 is withdrawn from the lumen of the injection cannula 194 and a source of injection fluid is connected in fluid flow communication with the proximal end 214 of the injection cannula 194. The injection fluid is injected through the lumen of the injection cannula 194 and exits out side ports 212 and enters the distraction device and possibly the resident volume of the vertebral body, where the fluid interdigitates with the cancellous bone tissue. The presence of the distraction device 250 advantageously substantially limits or prevents the fluid from entering any other region of the vertebral body, including extravasation into the vicinity of the spinal cord.

The injection cannula 194 may either be held stationary while injecting fluid into the treatment site or may be moved with respect to the distraction device 250. In one embodiment, the injection cannula 194 is moved proximally with respect to the distraction device 250 (i.e., withdrawn from the distraction device 250) while injecting fluid. By withdrawing the injection cannula 194 while injecting fluid into the treatment site, a different fluid dispersion profile can be achieved in the treatment site as compared to the dispersion profile achieved by injecting fluid while maintaining the injection cannula 194 in place.

In yet another embodiment, the injection cannula 194 may be moved to a location within the distraction device 250 and then held stationary while fluid is injected. When a suitable amount of fluid has been injected for that particular location, the injection cannula 194 may be moved to a different location within the distraction device 250 (e.g., by moving the injection cannula 194 proximally with respect to the distraction device 250) and then held stationary for a second fluid injection step. This process may be repeated multiple times for a fluid dispersion profile that is comparable to, but somewhat different from, the aforementioned simultaneous injection cannula 194 movement and fluid injection. For example, the injection cannula 194 may be positioned so as to inject fluid at three or more specific locations, such as: (1) at or adjacent to the distal end of the deployed distraction device 250, (2) at or adjacent to the middle of the deployed distraction device 250, and (3) at or adjacent to the proximal end of the deployed distraction device 250. By doing so, fluid will be injected near the inferior and superior endplates of the vertebral body, as well as at a more central location in the resident volume defined by the deployed distraction device 250. Other fluid injection profiles may also be employed without departing from the scope of the present disclosure.

Upon completion of fluid injection, the injection cannula 194 is withdrawn from the distraction device 250 while the distraction device 250 preferably remains in the vertebral body 106. Once the injection cannula 194 is removed from the patient, the small entrance incision may be sutured or otherwise cleaned and closed. While the fluid delivery device 192 of these figures is shown in use in the vertebral body with a distraction device implanted therein, it shall be appreciated that one skilled in the art could use the fluid delivery device 194 without a distraction device. More specifically, the injection cannula could be introduced within vertebral body 106 and moved as desired to deliver fluid.

While the foregoing methods illustrate how a fluid delivery device according to the present disclosure may be used to treat a fractured vertebral body, it should be understood that these are not the only procedures which may be practiced. In general, fluid delivery devices according to the present disclosure may be used for any procedure requiring fluid to be injected into a treatment site within a body, although it has particular application in treatment of VCF.

The following paragraphs include several examples of devices according to the present disclosure and additional aspects thereof.

A first example of a fluid delivery device for injecting fluid into an interior of a bone includes an elongated, hollow cannula having a proximal end portion; a distal end portion; a shaft portion between the proximal and distal end portions; and a fluid flow lumen extending through the shaft portion between the proximal and distal end portions. The distal end portion is flexible and may be flexed to assume a curved configuration having a radially inner side and a radially outer side. The radially inner side includes at least one aperture to direct fluid flowing from the lumen in a radially inward direction.

A second example of a fluid delivery device for injecting fluid into an interior of a bone includes an outer tubular member including a proximal end opening, a distal end opening, and a lumen extending therebetween. The device also includes an elongated, hollow cannula movably receivable within the lumen of the outer tubular member. The cannula includes a proximal end portion; a distal end portion; a shaft portion between the proximal and distal end portions; and a fluid flow lumen extending through the shaft portion between the proximal and distal end portions. The distal end portion of the cannula is flexible and constrained to maintain a generally straight configuration when the distal end portion is disposed within the outer tubular member and may be flexed to assume a curved configuration having a radial inner side and a radially outer side when the distal end portion is disposed outside of the outer tubular member. The radially inner side includes at least one aperture to direct fluid flowing from the fluid flow lumen in a radially inward direction.

In one aspect the present disclosure relates to the device of Examples 1 or 2 wherein the distal-most end of the lumen is closed.

In another aspect the present disclosure relates to the device of Examples 1 or 2 wherein the distal-most end of the cannula is pointed.

In yet another aspect the present disclosure relates to the device of Examples 1 or 2, alone or in combination with any of the aspects in paragraphs [00102] or [00103], wherein the radially outer side substantially blocks fluid flow from the lumen in a radially outward direction from the distal end portion.

A further aspect of the present disclosure relates to the device of Examples 1 or 2, alone or in combination with any of in paragraphs [00102] to [00104], wherein the radially outer side is substantially free of any fluid flow apertures therethrough communicating with the lumen.

Yet a further aspect of the present disclosure relates to the device of Examples 1 or 2, alone or in combination with any of the aspects in paragraphs [00102] to [00105], wherein the proximal end portion includes a connector for removably connecting the device to a source of fluid.

Another aspect of the present disclosure relates to the device of Examples 1 or 2, alone or in combination with any of the aspects in paragraphs [00102] to [00106], comprising a source of fluid, which fluid comprises at least one of a medication, drug, bone growth stimulant, bone replacement, poly(methyl methacrylate) material, stem cells, or bone cement.

Yet another aspect of the present disclosure relates to the device of Examples 1 or 2, alone or in combination with any of the aspects in paragraphs [00102] to [00107], wherein the at least one aperture comprises an elongated, longitudinally extending aperture.

Still yet another aspect of the present disclosure relates to the device of Examples 1 or 2, alone or in combination with any of the aspects in paragraphs [00102] to [00108], wherein the distal end portion comprises a plurality of apertures through the radially inner side.

An additional aspect of the present disclosure relates to the device of Examples 1 or 2, alone or in combination with any of the aspects in paragraphs [00102] to [00109], wherein the distal end portion is flexed by a predisposition to assume a spiral or helical configuration.

Another aspect of the present disclosure relates to the device of Examples 1 or 2, alone or in combination with any of the aspects in paragraphs [00102] to [00110], wherein the distal end portion is flexed by a predisposition to assume a circular configuration.

Yet another aspect of the present disclosure relates to the device of any of Examples 1 or 2, alone or in combination with any of the aspects in paragraphs [00102] to [00111], wherein at least the distal end portion comprises a shape memory material.

Still yet another aspect of the present disclosure relates to the device of Examples 1 or 2, alone or in combination with any of the aspects in paragraphs [00102] to [00112], wherein at least the distal end portion comprises a nickel titanium alloy.

A further aspect of the present disclosure relates to the device of Examples 1 or 2, alone or in combination with any of the aspects in paragraphs [00102] to [001113], wherein the distal end portion is flexed by a predisposition to curve about an axis extending at an angle, including up to about 90°, to the shaft portion.

An additional aspect of the present disclosure relates to the device of Examples 1 or 2, alone or in combination with any of the aspects in paragraphs [00102] to [00114], further comprising a laterally extending structure carried by the proximal end portion of the cannula, wherein the laterally extending structure defines a plane and the distal end portion of the cannula is predisposed to curve substantially within said plane.

Another aspect of the present disclosure relates to the device of Examples 1 or 2, alone or in combination with any of the aspects in paragraphs [00102] to [00115], wherein the cannula is pre-filled with an amount of fluid.

Yet another aspect of the present disclosure relates to the device of Examples 1 or 2, alone or in combination with any of the aspects in paragraphs [00102] to [00116], wherein the distal-most end of the cannula is pointed.

A further aspect of the present disclosure relates to the device of Example 1, alone or in combination with any of the aspects in paragraphs [00102] to [00117], further comprising an outer tubular member for slidably receiving the cannula therewithin, the tubular member constraining the distal end portion of the cannula in a generally straight configuration when the distal end portion of the cannula is disposed within the tubular member.

Yet a further aspect of the present disclosure relates to the device of Example 1, alone or in combination with any of the aspects in paragraphs [00102] to [00118], further comprising a locking mechanism associated with the outer tubular member which releasably holds the cannula in the cannula-constrained position.

A third example of a fluid delivery device for injecting fluid into an interior of a bone, includes an elongated, substantially hollow cannula including proximal and distal sections and an elongated guide member at least partially received within the cannula. The guide member includes a core wire and a coil spring surrounding the core wire. The proximal section of the cannula has a greater stiffness than the distal section of the cannula.

In one aspect the present disclosure relates to the device of Example 3, wherein the coil spring further comprises a distal end and the core wire is secured to the distal end of the coil spring.

In another aspect the present disclosure relates to the device of Example 3, wherein the core wire further comprises a distal end and at least a portion of the coil spring extends distally beyond the distal end of the core wire.

Another aspect of the present disclosure relates to the device of Example 3, alone or in combination with any of the aspects in paragraphs [00121] to [00122], wherein the core wire further comprises a proximal end and the coil spring is secured to the proximal end of the core wire.

Yet another aspect of the present disclosure relates to the device of Example 3, alone or in combination with any of the aspects in paragraphs [00121] to [00123], wherein the cannula further comprises an inner layer and an outer layer.

Still yet another aspect of the present disclosure relates to the device of Example 3, alone or in combination with the aspect in paragraph [00123], wherein the inner layer has a greater stiffness than the outer layer.

An additional aspect of the present disclosure relates to the device of Example 3, alone or in combination with any of the aspects in paragraphs [00121] to [00125], wherein the coil spring further comprises a distal end and an atraumatic cap is secured to the distal end of the coil spring.

Another aspect of the present disclosure relates to the device of Example 3, alone or in combination with any of the aspects in paragraphs [00121] to [00126], wherein the coil spring further comprises proximal and distal ends and the outer diameter of the distal end of the coil spring is less than the outer diameter of the proximal end of the coil spring.

Yet another aspect of the present disclosure relates to the device of Example 3, alone or in combination with any of the aspects in paragraphs [00121] to [00127], wherein the core wire is comprised of a metallic material.

Still yet another aspect of the present disclosure relates to the device of Example 3, alone or in combination with any of the aspects in paragraphs [00121] to [00128], wherein the coil spring is comprised of a metallic material.

An additional aspect of the present disclosure relates to the device of Example 3, alone or in combination with any of the aspects in paragraphs [00121] to [00129], further comprising an outer sheath surrounding at least a portion of the coil spring.

Another aspect of the present disclosure relates to the device of Example 3, alone or in combination with any of the aspects in paragraphs [00121] to [00130], further comprising an outer sheath entirely surrounding the coil spring.

Yet another aspect of the present disclosure relates to the device of Example 3, alone or in combination with any of the aspects in paragraphs [00130] or [00131], wherein the outer sheath has a lower friction than the coil spring.

Still yet another aspect of the present disclosure relates to the device of Example 3, alone or in combination with any of one of the aspects in paragraphs [00130] to [00132], wherein the outer sheath is comprised of a polymeric material.

An additional aspect of the present disclosure relates to the device of Example 3, alone or in combination with any of the aspects in paragraphs [00121] to [00133], wherein the core wire further comprises proximal and distal portions and the outer diameter of the distal portion is less than the outer diameter of the proximal portion.

Another aspect of the present disclosure relates to the device of Example 3, alone or in combination with any of the aspects in paragraphs [00121] to [00134], wherein the core wire further comprises a proximal portion, the coil spring further comprises a proximal end, and the outer diameter of the proximal portion of the core wire is approximately equal to the inner diameter of the proximal end of the coil spring.

Yet another aspect of the present disclosure relates to the device of Example 3, alone or in combination with any of the aspects in paragraphs [00121] to [00135], further comprising mating connectors on the cannula and the guide member for temporarily securing the cannula and the guide member.

Still yet another aspect of the present disclosure relates to the device of Example 3, alone or in combination with the aspect of paragraph [00132], wherein the guide member further comprises a proximal end and the mating connectors are associated with the proximal section of the cannula and the proximal end of the guide member.

A further aspect of the present disclosure relates to the device of Example 3, alone or in combination with any of the aspects in paragraphs [00136] or [00137], wherein the mating connectors comprise a luer cap and a luer hub.

A fourth example of a fluid delivery device for injecting fluid into an interior of a bone includes an elongated, substantially hollow cannula having proximal and distal sections and comprised of an inner layer and an outer layer, wherein the proximal section has a greater stiffness than the distal section and the inner layer has a greater stiffness than the outer layer.

In one aspect the present disclosure relates to the device of Example 4, wherein the distal section has an atraumatic end.

In another aspect the present disclosure relates to the devices of Examples 3 or 4, alone or in combination with any of the aspects in paragraphs [00124], [00125], or [00140], wherein the inner layer is comprised of a polymeric material.

In yet another aspect the present disclosure relates to the devices of Examples 3 or 4, alone or in combination with any of the aspects in paragraphs [00124], [00125], [00140] or [00141], wherein the outer layer is comprised of polymeric material.

In still yet another aspect the present disclosure relates to the devices of Examples 3 or 4, alone or in combination with any of the aspects in paragraphs [00124], [00125], or [00140] to [00142], wherein the inner layer is joined to the outer layer by a thermal processing procedure.

An additional aspect the present disclosure relates to the devices of Examples 3 or 4, alone or in combination with any of the aspects in paragraphs [00124], [00125], or [00140] to [00143], further comprising a reinforcing material associated with the proximal section.

Another aspect the present disclosure relates to the device of Example 4, alone or in combination with the aspect in paragraph [00144], wherein the reinforcing material is comprised of a metallic material.

Another aspect the present disclosure relates to the device of Example 4, alone or in combination with any of the aspects in paragraphs [00144] or [00145], wherein the reinforcing material is wound.

In yet another aspect the present disclosure relates to the device of Example 4, alone or in combination with any of the aspects in paragraphs [00144] or [00145], wherein the reinforcing material is braided.

In still yet another aspect the present disclosure relates to the device of Example 4, alone or in combination with any of the aspects in paragraphs [00144] or [00145], wherein the reinforcing material is woven.

An additional aspect the present disclosure relates to the device of Example 4, alone or in combination with any of the aspects in one of paragraphs [00144] to [00148], wherein the reinforcing material is positioned between the inner layer and the outer layer.

Another aspect the present disclosure relates to the device of Example 4, alone or in combination with any of the aspects in one of paragraphs [00144] to [00149], wherein the reinforcing material has a greater stiffness than the inner layer and the outer layer.

Another aspect the present disclosure relates to the device or aspects in one of paragraphs [00124] to [00150], wherein the cannula includes a plurality of longitudinally spaced injection ports for dispensing injection fluid.

Yet another aspect the present disclosure relates to the aspect in paragraph [00151], wherein at least two of said injection ports are differently sized to maintain substantially equal flow rates of injection fluid from said at least two of said injection ports.

Still yet another aspect the present disclosure relates to the aspect in paragraph [00151], wherein at least two of said injection ports are differently sized to maintain an unequal flow rate of injection fluid from said at least two of said injection ports.

An additional aspect the present disclosure relates to any one of the aspects in paragraphs [00151] to [00153], wherein the injection ports are configured to inject fluid in a direction generally transverse to a longitudinal axis of the cannula.

A fifth example of a fluid delivery device for injecting fluid into an interior of a bone, includes an elongated, substantially hollow cannula having proximal and distal sections, wherein the proximal section is substantially inflexible and the distal section is substantially flexible.

In one aspect the present disclosure relates to the device of Example 5, wherein the proximal section is comprised of a metallic material.

Another aspect of the present disclosure relates to the device of Example 5, alone or in combination with the aspect in paragraph [00156], wherein the distal section is comprised of a non-metallic material.

Yet another aspect of the present disclosure relates to the device of Example 5, alone or in combination with any of the aspects in paragraphs [00156] or [00157], wherein the distal section is comprised of a polymeric material.

A sixth example of a fluid delivery device for injecting fluid into an interior of a bone, includes an elongated, substantially hollow cannula configured to accommodate flow of an injection fluid therethrough at a flow rate, wherein the cannula includes a plurality of longitudinally spaced injection ports for dispensing injection fluid out of the cannula, at least two of said injection ports being differently sized to maintain substantially equal flow rates of injection fluid from said at least two of said injection ports.

In one aspect the present disclosure relates to the device of Example 6, wherein the cannula is generally flexible.

Another aspect of the present disclosure relates to the device of Example 6, alone or in combination with the aspect in paragraph [00160], wherein the injection ports are configured to inject fluid in a direction generally transverse to a longitudinal axis of the cannula.

Yet another aspect of the present disclosure relates to the device of Example 6, alone or in combination with any of the aspects in paragraphs [00160] or [00161], wherein at least a portion of the cannula is comprised of a polymeric material.

A seventh example of a device includes an elongated guide member movable with an injection cannula through the interior of an implant deployed in the body, comprising proximal and distal sections, wherein the proximal section is substantially inflexible and the distal section is substantially flexible.

In one aspect the present disclosure relates to the device of Example 7, wherein the proximal section is comprised of a metallic material.

Another aspect of the present disclosure relates to the device of Example 7, alone or in combination with the aspect in paragraph [00164], wherein the distal section is comprised of a non-metallic material.

Yet another aspect of the present disclosure relates to the device of Example 7, alone or in combination with any of the aspects in paragraphs [00164] or [00165], wherein the distal section is comprised of a polymeric material.

An eighth example of a device includes an elongated guide member movable with an injection cannula through the interior of an implant deployed in the body, comprising a core wire having a distal end; and a coil spring surrounding the core wire, wherein at least a portion of the coil spring extends distally beyond the distal end of the core wire.

In one aspect the present disclosure relates to the device of Example 8, wherein the coil spring is secured to the core wire.

Another aspect of the present disclosure relates to the device of Example 8, alone or in combination with the aspect in paragraph [00168], wherein the coil spring is secured to a proximal end of the core wire.

Yet another aspect of the present disclosure relates to the device of Example 8, alone or in combination with any of the aspects in paragraphs [00168] or [00169], further comprising an atraumatic cap secured to a distal end of the coil spring.

Still yet another aspect of the present disclosure relates to the device of Example 8, alone or in combination with any of the aspects in paragraphs [00168] to [00170], wherein the outer diameter of a distal end of the coil spring is less than the outer diameter of a proximal end of the coil spring.

An additional aspect of the present disclosure relates to the device of Example 8, alone or in combination with any of the aspects in paragraphs [00168] to [00171], wherein the core wire is comprised of a metallic material.

Another aspect of the present disclosure relates to the device of Example 8, alone or in combination with any of the aspects in paragraphs [00168] to [00172], wherein the coil spring is comprised of metallic material.

Yet another aspect of the present disclosure relates to the device of Example 8, alone or in combination with any of the aspects in paragraphs [00168] to [00173], further comprising an outer sheath surrounding at least a portion of the coil spring.

Still yet another aspect of the present disclosure relates to the device of Example 8, alone or in combination with any of the aspects in paragraphs [00168] to [00173], further comprising an outer sheath entirely surrounding the coil spring.

An additional aspect of the present disclosure relates to any one or combination of the aspects in paragraphs [00174] or [00175], wherein the outer sheath has a lower friction than the coil spring.

Another aspect of the present disclosure relates to any one or combination of the aspects in paragraphs [00174] to [00176], wherein the outer sheath is comprised of a polymeric material.

A ninth example of a device includes an elongated guide member movable with an injection cannula through the interior of an implant deployed in the body, comprising a coil spring; and a core wire at least partially received within the coil spring and including a proximal portion and a distal portion. The outer diameter of the distal portion is less than the outer diameter of the proximal portion.

In one aspect the present disclosure relates to the device of Example 9, wherein the core wire is secured to the coil spring.

In another aspect the present disclosure relates to the device of Example 9, alone or in combination with the aspect in paragraph [00179], wherein the coil spring further comprises proximal and distal ends and the core wire is secured to the proximal and distal ends of the coil spring.

Yet another aspect the present disclosure relates to the device of Example 9, alone or in combination with any of the aspects in paragraphs [00179] or [00180], wherein the coil spring further comprises a proximal end and the outer diameter of the proximal portion of the core wire is approximately equal to the inner diameter of the proximal end of the coil spring.

Still yet another aspect the present disclosure relates to the device of Example 9, alone or in combination with any of the aspects in paragraphs [00179] to [00181], wherein the coil spring further comprises a distal end and an atraumatic cap is secured to the distal end of the coil spring.

A further aspect the present disclosure relates to the aspect in paragraph [00182], wherein the distal portion of the core wire is secured to the atraumatic cap.

Another aspect the present disclosure relates to the device of Example 9, alone or in combination with any of the aspects in paragraphs [00179] to [00183], wherein the coil spring further comprises proximal and distal ends and the outer diameter of the distal end of the coil spring is less than the outer diameter of the proximal end of the coil spring.

Yet another aspect the present disclosure relates to the device of Example 9, alone or in combination with any of the aspects in paragraphs [00179] to [00184], wherein the core wire is comprised of a metallic material.

Still yet another aspect the present disclosure relates to the device of Example 9, alone or in combination with any of the aspects in paragraphs [00179] to [00184], wherein the coil spring is comprised of a metallic material.

Even still yet another aspect the present disclosure relates to the device of Example 9, alone or in combination with any of the aspects in paragraphs [00179] to [00185], further comprising an outer sheath surrounding at least a portion of the coil spring.

An additional aspect the present disclosure relates to the device of Example 9, alone or in combination with any of the aspects in paragraphs [00179] to [00186], further comprising an outer sheath entirely surrounding the coil spring.

Another aspect the present disclosure relates to any one or combination of the aspects in paragraphs [00187] or [00188], wherein the outer sheath has a lower friction than the coil spring.

Yet another aspect the present disclosure relates to any one or combination of the aspects in paragraphs [00187] or [00189], wherein the outer sheath is comprised of a polymeric material.

It will be understood that the embodiments and examples described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. A device for controlled direction of fluid delivery into a body, comprising:
an elongated, hollow cannula having
a proximal end portion;
a distal end portion;
a shaft portion between the proximal and distal end portions; and
a fluid flow lumen extending through the shaft portion between the proximal and distal end portions, wherein;
the distal end portion is flexible and may be flexed to assume a curved configuration having a radially inner side and a radially outer side; and
the radially inner side includes at least one aperture to direct fluid flowing from the lumen in a radially inward direction and the radially outer side substantially blocks fluid flow from the lumen in a radially outward direction.

2. The device of claim 1 wherein a distal-most end of the lumen is closed.

3. The device of claim 1 wherein the at least one aperture comprises one generally rectangular aperture in at least the distal end portion of the cannula.

4. The device of claim 1 wherein the distal end portion is flexed by a predisposition to assume the curved configuration.

5. The device of a claim 1 wherein the curved configuration is one of an arc, spiral or helical configuration.

6. The device of claim 4 wherein at least the distal end portion comprises a shape memory material.

7. The device of claim 4 wherein the distal end portion is flexed by a predisposition to curve about an axis extending at an angle, including up to about 90°, to the shaft portion.

8. The device of claim 1 further comprising a laterally extending structure carried by the proximal end portion of the cannula, wherein the laterally extending structure defines a plane and the distal end portion of the cannula is predisposed to curve substantially within said plane.

9. The device of claim 1 further comprising an outer tubular member for slidably receiving the cannula therewithin, the outer tubular member constraining the distal end portion of the cannula in a generally straight configuration when the distal end portion of the cannula is disposed within the outer tubular member.

10. The device of claim 1 wherein the cannula includes a plurality of longitudinally spaced injection ports for dispensing injection fluid, wherein at least two of the plurality of injection ports are differently sized to maintain substantially equal flow rates of injection fluid from said at least two of the plurality of injection ports.

11. A device for controlled direction of fluid delivery into a body comprising:
an elongated, substantially hollow cannula including proximal and distal sections, wherein the proximal section of the cannula has a greater stiffness than the distal section of the cannula; and
an elongated guide member at least partially received within the cannula and including a core wire and a coil spring surrounding the core wire.

12. The device of claim 11 wherein the cannula further comprises an inner layer and an outer layer, wherein the inner layer has a greater stiffness than the outer layer.

13. The device of claim 12 wherein the inner layer is joined to the outer layer by a thermal processing procedure.

14. The device of claim 12 wherein the cannula further comprises a reinforcing material associated with the proximal section.

15. The device of claim 14 wherein the reinforcing material has a greater stiffness than the inner layer and the outer layer.

16. The device of claim 11 wherein the cannula includes a plurality of longitudinally spaced injection ports for dispensing injection fluid in a direction generally transverse to a longitudinal axis of the cannula.

17. The device of claim 16 wherein at least two of the plurality of injection ports are differently sized to maintain substantially equal flow rates of injection fluid from said at least two of the plurality of injection ports.

18. The device of claim 11 wherein the coil spring further comprises proximal and distal ends and an outer diameter of the distal end of the coil spring is less than an outer diameter of the proximal end of the coil spring.

19. The device of claim 11 further comprising an outer sheath surrounding at least a portion of the coil spring, wherein the outer sheath has a lower coefficient of friction than the coil spring.

20. The device of claim 11 wherein the core wire further comprises proximal and distal ends and an outer diameter of the distal end of the core wire is less than an outer diameter of the proximal end of the core wire.
